SEQUENCE LISTING (4 sequences)

SEQ ID NO: 1 - protein, 127 aa, Homo sapiens
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 2 - protein, 21 aa, Homo sapiens
KVLDAVRGSPAINVAVHVFRK

SEQ ID NO: 3 - protein, 9 aa, Homo sapiens
AVHVFRKAA

SEQ ID NO: 4 - protein, 6 aa, Homo sapiens
VHVFRK

(19) United States
(12) Patent Application Publication
(10) Pub. No.: US 2015/0037341 A1
(43) Pub. Date: Feb. 5, 2015

(54) Diagnostic and Therapeutic Method

(71) Applicants: Ulf Lindahl, Uppsala (SE); Jin-ping Li, Uppsala (SE); Fredrik Noborn, Uppsala (SE)

(72) Inventors: Ulf Lindahl, Uppsala (SE); Jin-ping Li, Uppsala (SE); Fredrik Noborn, Uppsala (SE)

(73) Assignee: Plysackaridforskning i Uppsala AB, Uppsala (SE)

(21) Appl. No.: 14/000,683

(22) PCT Filed: Feb. 21, 2012
(86) PCT No.: PCT/EP2012/052919

§ 371 Date: Oct. 18, 2013

(30) Foreign Application Priority Data
Feb. 21, 2011 (FI) 20115165

(51) Int. Cl. A61K 31/727; G01N 33/68

(52) U.S. Cl. CPC A61K 31/727; G01N 33/6896; G01N 2333/47
USPC 424/139.1; 436/501; 435/7.72; 435/7.92; 514/56; 514/17.7; 536/21; 530/327; 530/387.9

(57) ABSTRACT

The present invention relates to a method of treating or preventing transthyretin amyloidosis, pharmaceutical composition for use in said treatment or prevention, as well as to a diagnostic method and a kit.

### FIELD OF THE INVENTION

The present invention relates to a method of treating or preventing transthyretin amyloidosis, pharmaceutical composition for use in said treatment or prevention, as well as to a diagnostic method and a kit.

### BACKGROUND OF THE INVENTION

Transthyretin (TTR) is a functional plasma protein composed of four 14 kDa subunits. TTR is synthesized mainly by the liver and functions as a transporter for retinol and thyroxine. In addition to its physiological functions, TTR is found as a main constituent of amyloid fibrils in several distinct clinical forms of amyloidoses, including familial amyloid polyneuropathy (FAP), familial cardiac amyloidosis and sporadic senile systemic amyloidosis (SSA). SSA is caused by the selective deposition of wildtype (WT) TTR fibrils in cardiac tissue, affecting almost 25% of the population above 80 years of age.

Around 100 amino acid mutations of TTR have been linked to familial forms of TTR amyloidoses. Studies have shown that TTR forms amyloid through a process that is initiated by tetramer destabilization. The destabilization results in accumulation of monomers which can misfold and aggregate into fibrillar structures. Considerable effort has been devoted to studying the amyloidogenicity of TTR mutations related to the familial forms of the disease, though the majority of TTR-related amyloidosis is represented by sporadic cases caused by aggregation and deposition of the otherwise stable WT protein.

Heparan sulfate (HS), a sulfated polysaccharide consisting of repeating units of glucosamine and glucuronic acid, is associated with amyloid deposits in a number of amyloidoses. Although accumulating evidence suggests that HS plays an important part in amyloid deposition, the precise role of HS in the pathology of amyloidosis remains unresolved.

Thus a need remains in the art for elaborating the role of HS in TTR amyloidosis, as well as for developing medicaments therefore.

### BRIEF DESCRIPTION OF THE INVENTION

The present invention is based studies, wherein co-deposition of HS with TTR in the cardiomyopathic heart was detected by immunohistochemical stainings. In vitro studies revealed that HS and heparin promoted TTR fibrillogenesis, an effect that correlated with the degree of sulfation of the polysaccharide, and was exerted through a specific binding sequence on TTR. This promoting effect was further examined in a Drosophila model that expresses human TTR. Thus, interaction of TTR with heparin/HS is size-dependent and selective, with a preference for polysaccharides with higher degrees of sulfation.

The present invention provides a method of diagnosing TTR amyloidosis. The method comprises the steps of (a) providing a sample of a bodily fluid from a human subject, (b) determining the concentration of TTR in said bodily fluid, (c) determining said human subject as suffering from or at high risk of developing TTR amyloidosis, if the TTR concentration determined in step (b) is higher than in a control sample.

The present invention also provides a kit for use in the above diagnostic method, comprising an anti-TTR antibody, and one or more reagents for detecting the expression level of TTR in a sample of a bodily fluid.

The present invention further provides a method of preventing, alleviating and/or treating transthyretin (TTR) amyloidosis in a patient in need thereof by administering an efficient amount of an agent capable of interfering with the interaction of TTR and heparan sulfate (HS).

Furthermore, the present invention relates to a medical use of an agent capable of interfering with the interaction between HS and TTR for preventing, alleviating and/or treating TTR amyloidosis.

Still further, the present invention provides a pharmaceutical composition comprising an agent capable of interfering with the interaction between HS and TTR, and a pharmaceutically acceptable carrier.

Specific embodiments of the invention are set forth in the dependent claims.

Other objects, embodiments, details and advantages of the present invention will become apparent from the following drawings, detailed description and examples.

### BRIEF DESCRIPTION OF THE FIGURES

In the following the invention will be described in greater detail by means of preferred embodiments with reference to the attached figures, in which

FIG. 1 shows microscopic images demonstrating co-localization of TTR amyloid and HS in the heart of cardiomyopathy. Mycocardial sections (15 μm thick) from a 70-year-old patient with reported cardiomyopathy stained with Congo red were viewed under polarized light (A) and an adjacent section was stained with Alcian blue (B). Arrows in (A) and (B) indicate adjacent locations. Additional sections were double immunostained with antibodies against TTR (C) and HS (D). Merging the fluorescent channels from (A) and (B) shows overlapped positive signals for TTR and HS in patient specimen (E) and negative staining of TTR in age-matched control subject (F). DAPI counter-staining for nuclei (blue). Original magnification: A-F: 200×, Scale bar: 50 μM.

FIG. 2 demonstrates pH-dependent fibrillization of wildtype TTR. TTR (50 μM) was dissolved in phosphate-citric acid buffer at the pH-value as indicated and incubated at 37° C. for 48 h. After incubation the samples were analyzed for their fibril content using ThT fluorescence (A). Fibril formation was only detected in the samples incubated at pH 2.7, in a much smaller amount (200 AU) in comparison with the samples incubated in the presence of heparin (8000 AU; FIG. 3A) at the same pH. TTR (500 μM) was incubated under the same conditions as above and were analyzed by native-PAGE (B). The strong bands migrated to the bottom of the gel represent mono- and di-mers.

FIG. 3 shows the effect of heparin and HS on WT TTR aggregation. (A, B) The TTR dissolved in phosphate buffer (50 μM, pH 2.7) was incubated alone or with different heparin or HS (12 μM) at 37° C. for 48 h. After incubation the samples were mixed with ThT and fluorescence was measured. Data shown represent triplicate experiments (means±SEM). (C) TTR (500 μM) incubated with heparin (0, 5 or 20 μM) under the same conditions were analyzed with native-PAGE as described in Methods. The gel shows a different migration pattern of TTR incubated with or without heparin. The fibrils migrated in the stacking gel is not shown. (D) Microscopic analysis of the TTR after incubation with heparin. The sample was spotted on a glass and stained with Congo red. Images were taken under fluorescent light (Original magnification: 20×; Scale bar: 5 μm) and polarized light (insert).

FIG. 4 demonstrates heparin integration into TTR fibrils. TTR dissolved in phosphate buffer (pH 2.7) was co-incubated with heparin (mixture of ³H-labeled and non-labeled) at 37° C. for 48 h. (A) The TTR-heparin co-incubated sample and heparin alone were separated with native-PAGE and stained with Alcian blue. (B) An identical set of samples was separated on the native-PAGE as above, and each lane was cut into six sections (as illustrated in A). The gel slices were incubated for 2 hr in H₂O to extract the heparin. The radioactivity was measured by scintillation counting.

FIG. 5 shows an analysis of heparin incubated with pre-aggregated TTR. TTR dissolved in phosphate buffer (pH 2.7) was incubated without heparin at 37° C. for 48 h. Heparin (mixture of ³H-labeled and nonlabeled) was then added and the TTR-heparin sample was incubated for an additional hour at 37° C. The sample was thereafter separated by native-PAGE and the gel was cut into 6 sections as illustrated in FIG. 4A. Radioactivity in each section was measured by scintillation counting. Data represents triplicate experiments (mean±SEM).

FIG. 6 demonstrates HS/heparin binding to TTR at mild acidic conditions. (A) Dissociated TTR was immobilized onto a Biacore sensor chip and heparin (10 μM) was allowed to interact with the immobilized TTR in neutral or in mildly acidic conditions. (B) HS II (10 μM) binding to TTR at different pH was monitored and the response for each sample is expressed as response units (RU). The experiment was repeated three times and the graph shows a representative result. (C) TTR (50 μM) incubated with heparin or HS II for 7 days at pH 5.0 or pH 7.4 was analyzed by ThT fluorescence. Data represent triplicate experiments (means±SEM).

FIG. 7 demonstrates the binding affinity of heparan sulfate (HS) and heparin for TTR. Dissociated TTR was immobilized onto a Biacore sensor chip. Heparin (A), HS II (B) or HS I (C) were injected in a concentration series (ranging from 0.001-70 μM) over the surface at pH 5.0. The response for each concentration was measured at the steady-state region of the curve. The apparent equilibrium dissociation constant (KD) was calculated by plotting the response at steady-state for each concentration versus heparin/HS concentration. The data were fitted to the equation: Response=(Rmax*C)/(KD+C), where C is the concentration of heparin/HS. Two analyte flow rates (20 and 30 μl/min) were tested to assess possible effects of mass transport limitations in the system. Altering the flow rate had no significant effect on the obtained KD-value, indicating that mass transport effects were not a limiting factor in the assay. The presented values are average value of three independent experiments. (D) Thioflavin T (50 μM) injected over the surface immobilized with dissociated TTR resulted in no binding. In contrast, Thioflavin T injection over the surface immobilized with aggregated TTR revealed significant binding.

FIG. 8 demonstrates identification of HS/heparin binding sequence in TTR. (A) TTR amino acid sequence where histidine “(+)” and other basic amino acids “+” are marked. (B) Equal mole of TTR-peptides or full-length protein (with or without acidic dissociation), were incubated with ³H-labeled heparin (1500 cpm) at pH 5.0. The amount of ³H-labeled heparin bound to the peptides was analyzed using a nitrocellulose-filter trapping method.

FIG. 9 shows SPR analysis of TTR peptides binding to heparin. Heparin was immobilized onto a Biacore sensor chip (CM5) using the surface thiol coupling kit (GE Healthcare). Peptides derived from regions on the TTR sequence (FIG. 5A) containing a histidine residue (24-35, 51-61 and 84-94), were allowed to interact with the heparin surface at pH 5.0 (peptide conc. 25 μg/ml; flow rate 20 μl/min). In consistence with the result analyzed by the nitrocellulose filter-binding assay (FIG. 5B), only the peptide of aa 24-35 interacted with heparin (red).

FIG. 10 shows reduced HS binding activity to native TTR. (A) Equal amount of dissociated or native protein was immobilized onto a Biacore sensor chip and HS II was allowed to interact with the immobilized TTR at pH 5.0. (B) The values of the HS II-TTR interaction signal were obtained by repeated experiments using three chips with different ligand densities (250-3000 RU). The signals were normalized to a ligand density of 1000 RU and average value is shown. Statistical analysis was done by t test.

FIG. 11 shows the effect of HS on TTR fibril formation in cell culture. Dissociated TTR was added to WT (CHO-WT) and HS-deficient (CHO-pgsD-677) cells and cultured for 48 hr. (A) After extensive washing with PBS, the cells were lysed in 1% NaOH to extract cell-associated TTR fibrils. The lysed material was mixed with ThT and the fluorescence was measured. Values represent triplicate experiments (means±SEM). Statistical analysis was done by t test. (B) Cells were fixed and immunostained with anti-TTR antibody (HPA002550; green). DAPI counterstaining is shown in blue. Original magnification: 20×, scale bar: 20 μM.

FIG. 12 shows the effect of heparin on TTR fibril formation in vivo. (A) Eggs of TTR transgenic flies were cultured on regular medium (control) or medium supplemented with low molecular weight heparin (Klexane®) or heparin. Heads were collected 9-17 days posteclosion and lysed in saline. The extract was analyzed by ThT staining. Values represent the average of duplicate experiments. (B) The presence of heparin in the Drosophila head lysate was analyzed with dot-blot stained Alcian blue. Heparin (25 ng and 50 ng) was used as positive control. Twenty-five microliters of the lysate (same samples as used for ThT analysis) was applied to a nylon membrane (Hybond N+, GE Healthcare) and stained with Alcian blue (0.5% Alcian Blue dissolved in 25% isopropanol and 3% acetic acid).

FIG. 13 demonstrates heparin/HS binding domain on native TTR. A model of a TTR dimer, generated by the PyMol program (PDB accession 1 DVQ, (1)), showing the heparin/HS binding site on native TTR. The cluster of basic amino acids (31-HVFRK-35) in this domain, which are expected to be critical for TTR-heparin/HS binding, is marked in magenta. The binding site is partly embedded in the structure of native TTR tetramer form, providing a rational for the low HS binding activity of native TTR.

FIG. 14 illustrates the detection of heparin-TTR co-localization in TTR transgenic flies (A-G) Confocal microscopy analysis of cryo-sections prepared from Drosophila heads. (A) Overview image of a Drosophila head stained with DAPI (blue), showing the brain (Br), optical lobes (OI) and retina (Re). Sections prepared from transgenic flies cultured on standard medium (B) or heparin-supplemented medium (C) were stained with anti-TTR antibodies (red). (D) Section prepared from a transgenic fly cultured on heparin supplemented medium was stained with antibodies against TTR (red) and heparin (green). Co-deposition of TTR and heparin were observed in the retina. (E) An enlarged 3D-view generated by a z-stack from the marked area in (D), showing merged staining of TTR and heparin. (F) Staining for TTR and heparin in a section prepared from transgenic flies reared on regular medium and (G) a section from non-transgenic flies fed on heparin-supplemented medium Original magnifications: A 10×; B, C 20×; D-G 63×. Scale bars are indicated.

FIG. 15 illustrates the effect of heparin/HS on TTR aggregation. (A) Tetramer destabilization (promoted by acidic condition) leads to formation of monomers on which the heparin/HS binding domain is exposed. Mild acidic condition facilitates TTR binding to heparin providing a scaffold to promote fibril formation. (B) A hypothetical mechanism of TTR amyloidosis in the cardiac tissues. Possible change (increase of sulfation degree) in HS structure in the aged heart (left) in comparison to the normal HS structure in younger individuals (right) may promote TTR-HS interactions, leading to the accumulation and deposition of TTR in the heart associated with aging.

FIG. 16 demonstrates that an antibody raised against aa 24-35 of TTR is specific for the monomeric, i.e. dissociated form of TTR as opposed to the tetrameric, i.e. native form of TTR.

FIG. 17 demonstrates that the antibody recognized the native TTR peptide 24-35, but not a denatured form thereof. The figure also shows that the antibody recognizes TTR in human plasma.

FIG. 18 illustrates the effect of HS on TTR internalization in cell culture. Dissociated TTR (5 μM) was added to WT (CHO-WT) and HS-deficient (CHO-pgsD-677) cells and cultured for 48 hrs in F12 starvation medium (Gibco). Cells were stained with an anti-TTR antibody (light grey) and with DAPI for nucleus staining. (a) TTR-positive signal was found associated with the nucleus for CHO-WT cells. (b) An enlarged view of the marked area in panel a. (c) The CHO-pgsD-677 cells were absent of TTR-staining. Images were captured with fluorescence microscopy. Original magnification, 40×. Scale bars are indicated.

FIG. 19 illustrates a rapid internalization of dissociated TTR that may be detected within the nucleus of wild type cells. Dissociated TTR (5 μM) was added to WT (CHO-WT) and HS-deficient (CHO-pgsD-677) cells and cultured for 1.5 hrs in F12 starvation medium (Gibco). Cells were stained with an anti-TTR antibody (light grey) and with DAPI for nucleus staining. (a) TTR is observed in the perinuclear region of CHO-WT cells. (b) An enlarged view of the marked area in panel a. (c) Confocal z-scan images of 1 μm thickness were taken of the marked area in panel b. The images were processed into a 3D model using Imaris Bitplane imaging software. (d) The CHO-pgsD-677 cells were absent of TTR-staining. Images were captured with confocal scanning fluorescence microscopy. Original magnification, 20×. Scale bars are indicated.

FIG. 20 illustrates that aggregated TTR associates with wild type cells. Dissociated TTR (5 μM) was added to WT (CHO-WT) and HS-deficient (CHO-pgsD-677) cells and cultured for 1.5 hrs in F12 starvation medium (Gibco). Cells were stained with an anti-TTR antibody (circled area) and with DAPI for nucleus staining. (a) A representative image of CHO-WT cells incubated with aggregated TTR. (b) An enlarged view of the marked area in panel a. (c) The CHO-pgsD-677 cells were absent of TTR-staining. Images were captured with confocal scanning fluorescence microscopy. Original magnification, 20×. Scale bars are indicated.

FIG. 21 demonstrates that heparin binds to aggregated TTR. Recombinant wild type TTR (500 μM) was dissolved in phosphate buffer (pH 7.4 or 2.7) and incubated at 37° C. for the time period as indicated. (a) The incubated samples were analyzed for their degree of aggregation with native PAGE. (b) The samples were incubated with ³H-labelled heparin for 30 min at room temperature. The amount of heparin bound to the samples with different aggregation degree was analyzed by a nitrocellulose-filter trapping method.

### DETAILED DESCRIPTION OF THE INVENTION

The present invention is based on studies aimed at determining whether heparin/heparan sulfate (HS) plays a role in the pathogenesis of transthyretin (TTR)-related amyloidosis.

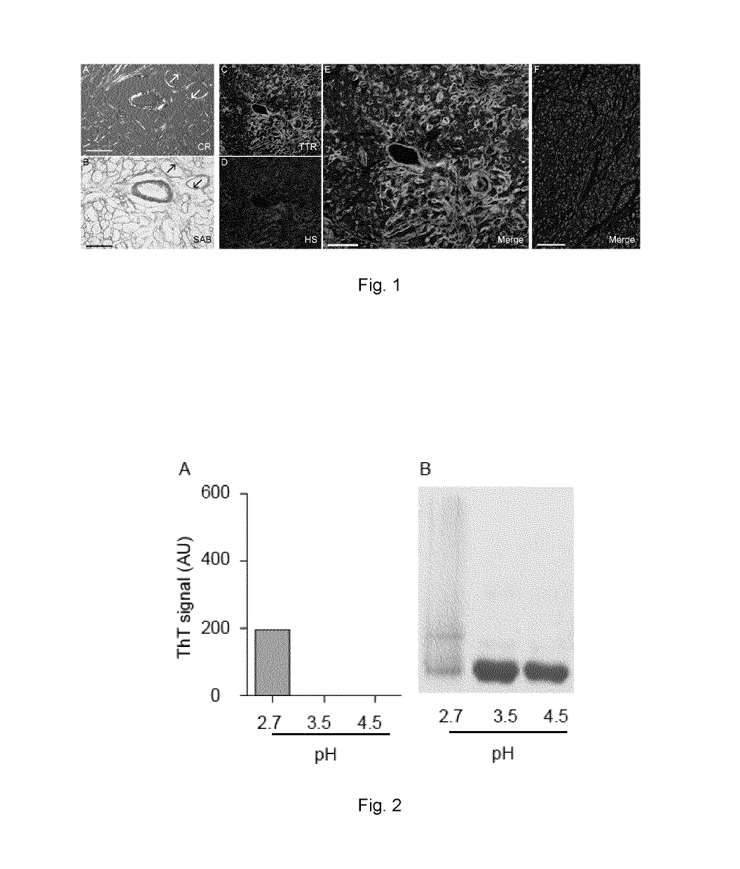

Immunohistological examination of a heart specimen from a patient diagnosed with amyloid cardiomyopathy revealed substantial HS accumulation with TTR amyloid deposits (FIG. 1), although some of the TTR positive signals, largely in vessel walls, did not overlap with HS. This finding of a high degree of HS-TTR co-localization prompted us to explore the potential functions of HS in TTR amyloidosis. To address this question, we employed recombinant human WT TTR to assess its aggregation in the absence or presence of heparin/HS by in vitro and in vivo experiments.

It has been established that TTR aggregation/amyloidosis is initiated by dissociation of the native tetramers, generating unstable monomers that spontaneously form oligmers. In agreement with earlier reports, we confirmed dissociation of the recombinant WT TTR upon incubation at pH 2.7, as analyzed by gel electrophoresis. Formation of amyloid-like fibrils was quantified by ThT fluorescence staining and the formation of oligomers was assessed by native-PAGE. We found that the WT TTR spontaneously aggregated to some extent upon incubation at 37° C. for 48 h at pH 2.7, but not at higher pH (FIG. 2). However, prolonged incubation under mildly acidic conditions resulted in aggregation of the TTR (FIG. 6C), indicating altered kinetics of TTR aggregation at different pH conditions. Without being limited to any theory, this may reflect the in vivo situation, where repeated shortage of oxygen supply in a cardiomyopathic heart provides an acidified environment that may facilitate TTR aggregation.

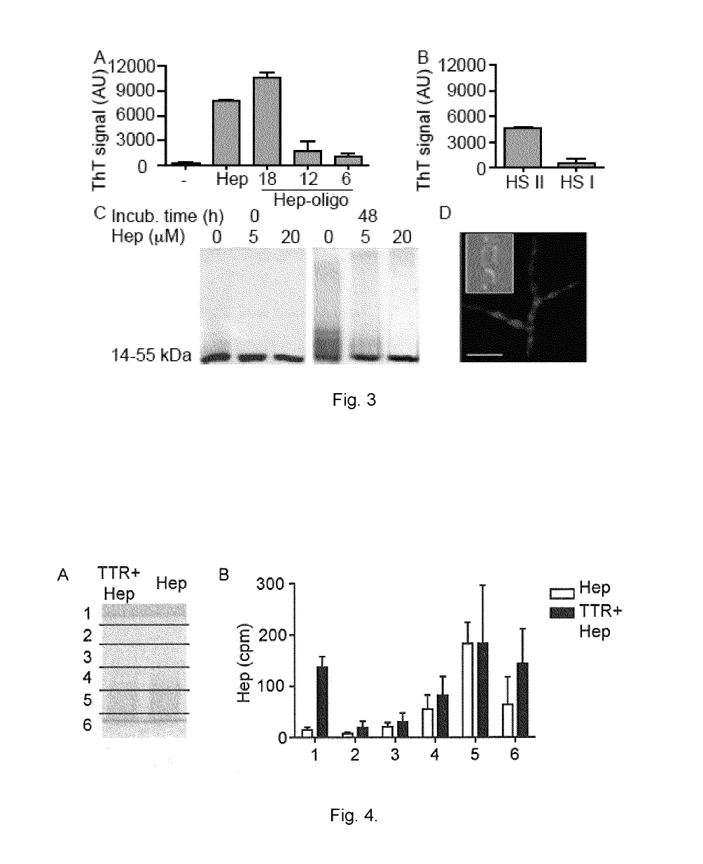

Addition of heparin, a commonly used analogue of HS, greatly promoted TTR fibrillization at pH 2.7 and 5.0 (FIGS. 3A and 6C). Notably, inclusion of heparin resulted in a differential pattern of TTR aggregates as demonstrated by native-PAGE analysis. Substantial quantities of macromolecules remained at the top of the gel reflecting a reduction or absence of oligomeric species (FIG. 3C). It is possible that heparin altered the rate of monomer/oligomer fibril assembly, or redirects the aggregation pathway.

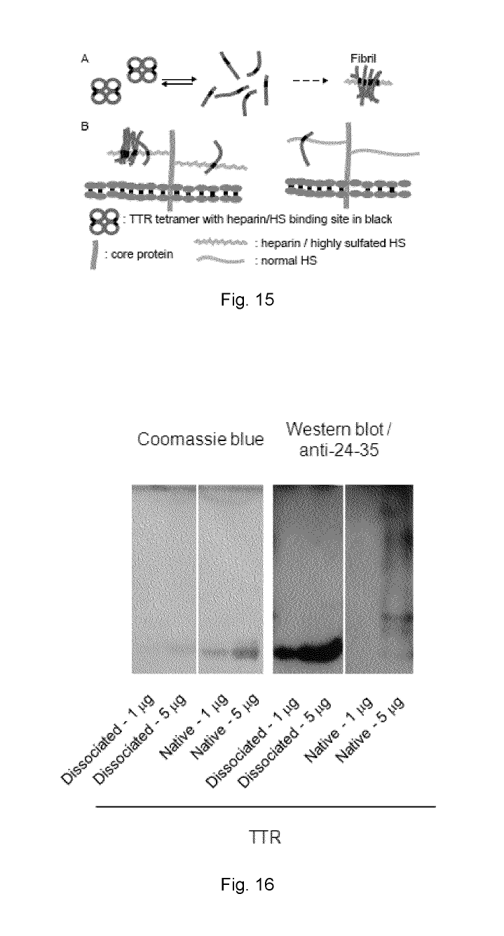

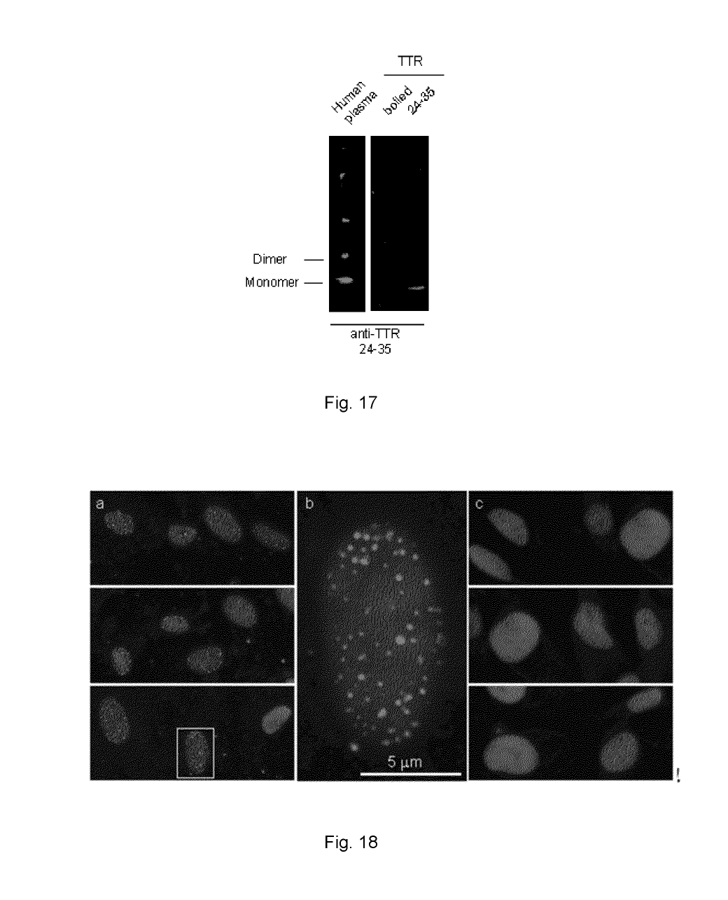

The finding that heparin is incorporated into the TTR fibrils (FIG. 4) suggests that HS/heparin can play a scaffold role, ‘collecting’ misfolded TTR monomers and being incorporated into the fibrils. Without being limited to any theory, this may reflect the in vivo TTR-HS aggregation where HS on the cell surface or in the extracellular matrix functions as a scaffold/catalyst to promote accumulation and aggregation of the unfolded TTR monomers. The scheme in FIG. 15 illustrates this hypothesis of HS-mediated TTR fibrillization.

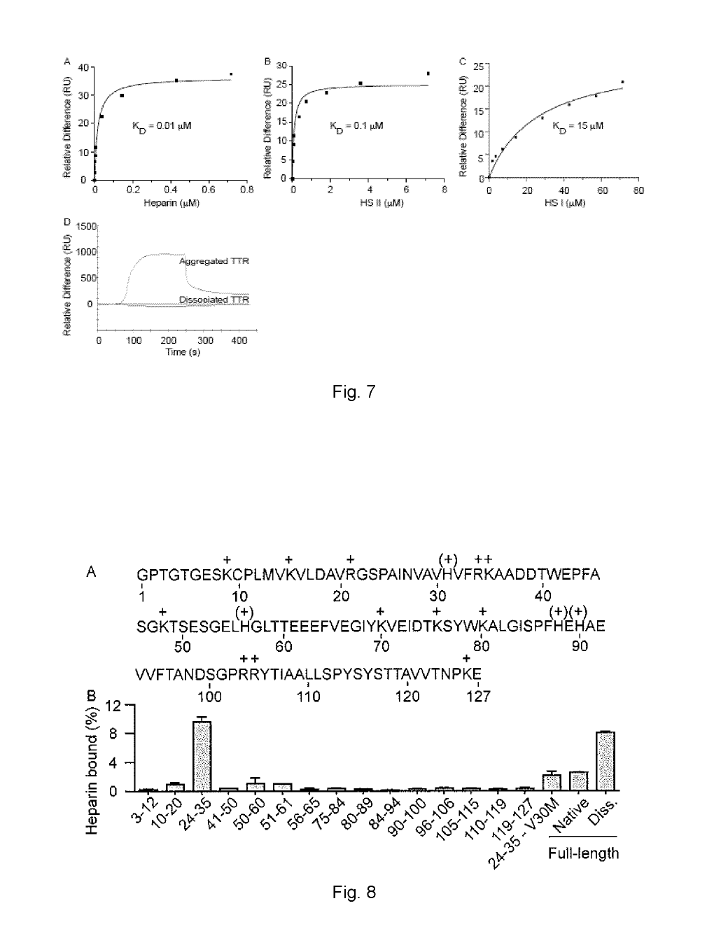

HS also promoted TTR fibrillogenesis, but to a lower degree compared to heparin (FIGS. 3A and 3B), in agreement with a recent report. Interestingly, the promoting ability of HS is associated with degree of sulfation. HS I that contains 5% trisulfated disaccharides had a marginal effect on TTR aggregation, while the highly sulfated HS II, containing 45% of the trisulfated component, had a stronger effect on TTR aggregation. A comparison of the TTR binding to heparin or the two HS preparations revealed that the difference in affinity was correlated with sulfation degree. TTR displayed a 10-fold higher affinity for heparin when compared to HS II and more than a 1000-fold higher affinity when compared to low sulfated HS I (FIG. 7). However, previous results have shown that chondroitin sulfate fails to promote aggregation of amyloid peptides, suggesting that the fine structure of HS is also important for the effect.

Intriguingly, the degree of HS sulfation has been reported to be age-dependent, as HS in the aorta of elderly individuals was found to be more sulfated than HS from young subjects. Concomitantly, aortic deposition of amyloid has been found in all investigated subjects above 80 years of age. This may suggest an association of age-dependent alteration in HS sulfation with aortic amyloidosis. Whether the high incidence of WT TTR amyloidosis in the heart of elderly is associated with a similar alteration of HS structure remains to be determined.

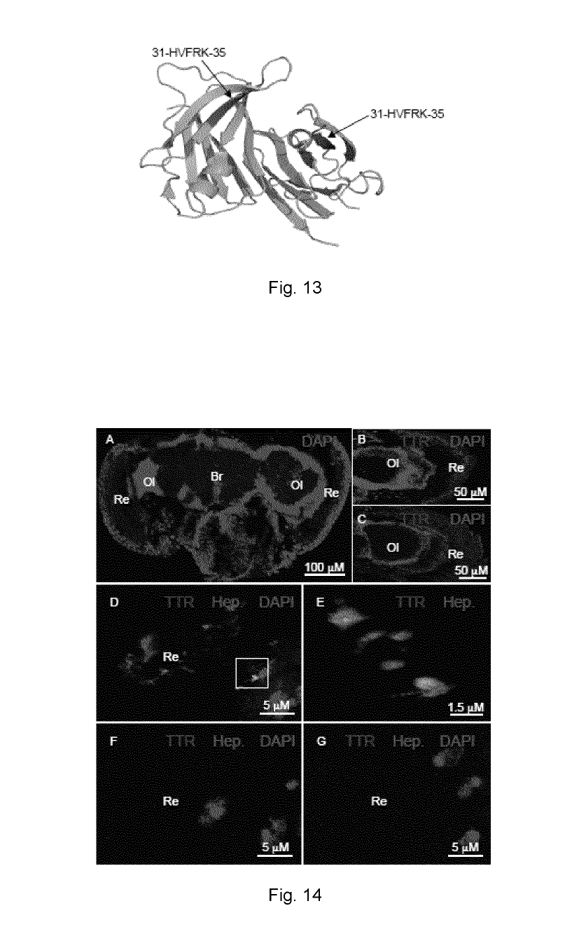

A long-asked question is why WT TTR forms amyloid, despite its well-demonstrated thermodynamic stability. To this end, our results showing that the sequence of aa 24-35 in WT TTR binds effectively to heparin may offer an explanation. In the native form of TTR this peptide is cryptic (FIG. 13) and incapable to interact with HS. When the protein is dissociated, the sequence becomes exposed, facilitating the interaction with HS. The basic property of the peptide, having 3 basic amino acids in close proximity (31-HVFRK-35), accommodates binding to the negatively charged HS/heparin through electrostatic interaction. This is of interest as similar HS/heparin binding motifs have been identified for other amyloid peptides, such as amyloid-beta (13-HHQK-16) and serum amyloid A (34-KYFHARGN-41).

Moreover, our finding that heparin binds better to the WT peptide (aa 24-35) than to the corresponding peptide containing the V30M mutation (associated with familial amyloid polyneuropathy) (FIG. 8B) may suggest a potential mechanism for the so far poorly understood phenomenon that V30M mutant and WT TTR displays a difference in tissue-specific deposition. Whether this different deposition pattern is correlated to the HS-binding property of WT v.s mutant TTR should be further investigated. Examination of HS structures from the amyloid deposits of WT and V30M TTR could be valuable to establish a correlation of TTR deposition with tissue-specific HS expression.

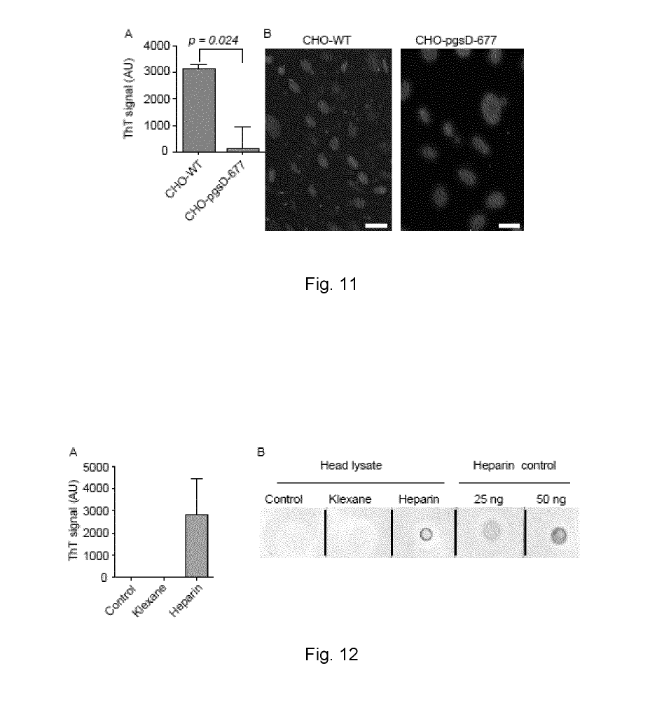

Furthermore, we wanted to know whether the effect of HS/heparin on TTR fibrillization could be reproduced in a cell model. Incubation of the TTR with WT CHO cells that express HS generated substantial amounts of amyloid-like fibrils; in comparison, the HS-deficient CHO-pgsD-677 cells only produced minor amounts of fibrils (FIG. 11). This result, combined with the strong TTR immunostaining in CHO-WT cells, clearly demonstrates a potential role for cellular HS in TTR fibrillization. This effect has further been demonstrated in a Drosophila model that expresses an amyloidogenic TTR. Feeding the animals with heparin promoted formation of TTR amyloid-like fibrils, a result not observed in the flies fed with heparin fragments (Klexane®). Correspondingly, immunohistostaining with antibodies against TTR and HS confirmed the co-deposition of TTR and heparin.

It should be noted that the 18-mer heparin fragment (Mr of 4,500 Da) promoted the in vitro TTR aggregation better than full-length heparin (FIG. 3A), while Klexane® (with an average Mr of 4,500 Da) failed to promote TTR fibrillization in vivo. Without being limited to any theory, this apparent discrepancy may be due to the molecular heterogeneity of Klexane®. The smaller fragments in the preparation may have interfered with TTR aggregation in the fly model; while the heparin 18-mer used in in vitro experiment is a homogenous preparation. In fact, low molecular weight heparin and heparin-like fragments have previously been shown to inhibit amyloidogenesis of SAA and amyloid-beta peptide. This raises the possibility that an optimal size of heparin will be able to interfere with the TTR-HS interaction, and attenuate TTR aggregation.

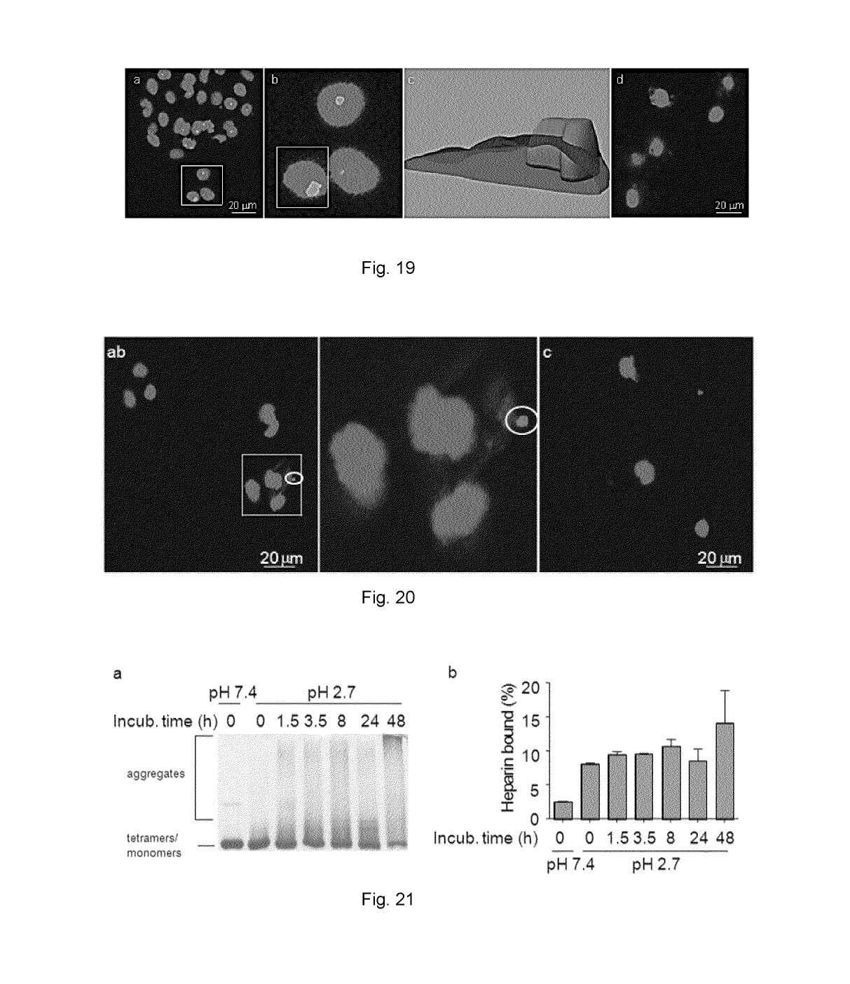

The finding suggests that HS may play dual roles in modulating the TTR metabolism. The HS expressed on the cell surface, such as macrophages, promote internalization of the monomeric form of TTR to prevent its aggregation (FIG. 19); the HS expressed on extracellular matrix may function as a scaffold to transfer the toxic oligomers formed to more stable fibrils. (FIG. 20).

In summary, information about pro-amyloidogenic factors in WT TTR fibrillization is scarce. As the majority of TTR-related amyloidosis is sporadic cases caused by WT TTR deposition, understanding the pathologic mechanisms of WT TTR deposition is of valuable for prevention and treatment of SSA. Our data provide the first evidence of codeposition of HS with WT TTR in a cardiomyopathic heart, and illustrates a proamyloidogenic role for HS/heparin in TTR aggregation through selective binding to a specific domain of TTR. The results suggest a potential mechanism by which HS influences TTR aggregation/amyloidosis in the heart of the elderly population. The findings are consistent with reports involving other amyloid precursors, and collectively may assist in the design of potential therapeutic agents for amyloid associated diseases.

Based on the present findings, TTR amyloidosis including, but not limited to, familial amyloid polyneuropathy (FAP), familial cardiac amyloidosis, and sporadic senile systemic amyloidosis (SSA) may be prevented, alleviated, ameliorated and/or treated by interfering with the interaction of HS with TTR. This may be achieved by administering to a human patient in need of such prevention, alleviation, amelioration and/or treatment an efficient amount a HS/heparin-derived fragment or mimetic, a TTR-derived peptide or a modified peptide or an anti-TTR antibody having the ability to interfere with the interaction between HS and TTR.

By an “efficient amount” of an agent capable of interfering with the interaction between HS and TTR is meant a level in which the harmful effects of TTR are, at a minimum, ameliorated. In other words, an efficient amount of such an agent is one that is sufficient to block, or partially block, the binding of HS with TTR, where such binding is harmful or undesired. Amounts and regimens for the administration of agents capable of interfering with the interaction between HS and TTR can be determined readily by those with ordinary skill in the clinical art of treating amyloidoses. Generally, the dosage of such interfering agents will vary depending on considerations such as: age, gender and general health of the patient to be treated; kind of concurrent treatment, if any; frequency of treatment and nature of the effect desired; extent of tissue damage; duration of the symptoms; and other variables to be adjusted by the individual physician. A desired dose can be administered in one or more applications to obtain the desired results.

Agents interfering with the physical interaction of HS and TTR may be formulated as pharmaceutical compositions comprising one or more pharmaceutically acceptable carrier and/or excipient. In some embodiments, the pharmaceutical composition is provided as unit dosage forms.

The present pharmaceutical compositions may be administered by various routes including but not limited to oral delivery, intravenous, intraperitoneal, subcutaneous, transdermal, topical and local administration.

For the purposes of parenteral or topical administration, the pharmaceutical composition may be formulated, for instance, as solutions, suspensions or emulsions. The formulations may comprise sterile aqueous or non-aqueous solvents, co-solvents, solubilizers, dispersing or wetting agents, suspending agents and/or viscosity agents, as needed. In some embodiments, an agent interfering with the interaction between HS and TTR is dissolved in sterile water for injection and the pH preferably adjusted to about 6 to 8 and the solution is preferably adjusted to be isotonic.

In some embodiments, the pharmaceutical composition may be provided in concentrated form or in form of a powder to be reconstituted on demand. In case of lyophilization, certain cryoprotectants are preferred, including polymers (povidones, polyethylene glycol, dextran), sugars (sucrose, glucose, lactose), amino acids (glycine, arginine, glutamic acid) and albumin. If solution for reconstitution is added to the packaging, it may consist e.g., of pure water for injection or sodium chloride solution or dextrose or glucose solutions.

Solid dosage forms for oral administration include capsules, tablets, pills, troches, lozenges, powders and granules. In such solid dosage forms, the active compound may be admixed with at least one inert diluent such as sucrose, lactose or starch. Such dosage forms may also comprise, as is normal practice, pharmaceutical adjuvant substances, e.g. stearate lubricating 35 agents or flavouring agents. Solid oral preparations can also be prepared with enteric or other coatings which modulate release of the active ingredients.

Liquid dosage forms for oral administration include pharmaceutically acceptable emulsions, solutions, suspensions, syrups and elixirs containing inert non-toxic diluents commonly used in the art, such as water and alcohol. Such compositions may also comprise adjuvants, such as wetting agents, buffers, emulsifying, suspending, sweetening and flavouring agents.

Means and methods for formulating the present pharmaceutical preparations are known to persons skilled in the art, and may be manufactured in a manner which is in itself known, for example, by means of conventional mixing, granulating, dissolving, lyophilizing or similar processes.

In some embodiments, an agent capable of interfering with the interaction of HS and TTR is a heparin/HS fragment or a mimetic thereof. Suitable heparin/HS molecules include, but are not limited to, disaccharides, tetramers, hexamers, octamers, decamers, 12-mers, 14-mers, 16-mers and 18-mers of non-anticoagulant heparin. However, in some embodiments, full-length non-anticoagulant heparin may be used. Furthermore, the sulfation degree of heparin/HS polymers, oligomers or fragments may vary between tri-sulfated and mono-sulfated mono-sugars. In some embodiments, the therapeutic efficiency of non-coagulant heparin may vary such that the higher the sulfation degree the higher the therapeutic efficiency.

The term “heparin/HS mimetic” refers to a sulfated oligosaccharide similar to but different from heparin/HS. Such oligosaccharides include, but are not limited to dextran sulfate, chondroitin sulfate, sulfated K5 polysaccharide, sulfated natural poly- and oligosaccharids (from plants and marine) as well as synthetic sulfated oligosaccharides. Again, the structure, including length and the level of sulfation may vary in different embodiments of the present invention.

In other embodiments, an agent capable of interfering with the interaction of HS and TTR is a TTR-derived peptide or a modified peptide thereof. Such peptides include those comprising or consisting of an amino acid sequence depicted in SEQ ID NO. 1, peptides comprising or consisting of amino acids 24-35 of SEQ ID NO. 1, those comprising or consisting of amino acids 50-61 or 10-24 of SEQ ID NO. 1, and those comprising or consisting of an amino acid sequence KVLDAVRGSPAINVAVHVFRK (SEQ ID NO. 2), preferably those comprising or consisting of an amino acid sequence AVHVFRKAA (SEQ ID NO. 3), and more preferably those comprising or consisting of an amino acid sequence VHVFRK (SEQ ID NO. 4), and any combinations of said peptides. Modified peptides suitable for use in the present invention include those having at least one modification in their amino acid sequence as compared to the sequence depicted in SEQ ID No. 1 or fragments thereof and retaining an ability to interfere with the interaction between HS and TTR. In other words, suitable peptides for use in the present invention include those having at least 80%, preferably at least 85%, 90%, 95%, 96%, 97%, 98% or 99% sequence identity with the amino acid sequence of TTR depicted in SEQ ID NO. 1 provided that they still have the ability to interfere with the interaction between TTR and HS.

In still other embodiments, an agent capable of interfering with the interaction of HS and TTR is a blocking antibody, preferably an antibody specifically binding to amino acids 24-35 of TTR as depicted in SEQ ID NO. 1., or to amino acids 50-61 or 10-24 of SEQ ID NO. 1, or any combinations thereof. Therapeutic anti-TTR antibodies may be produced by any standard method known in the art. Such antibodies may be a chimeric, humanized, fully human or recombinant antibodies. In some preferred embodiments, the antibody is a monoclonal antibody. In some other preferred embodiments, antibody fragments such as Fab, Fab′, F(ab′)2, FV or single chain FV fragments may be used.

An anti-TTR antibody for use in the present invention may be conjugated, either chemically or by genetic engineering, to other agents, which provide targeting of the antibodies to a desired site of action. Alternatively, other compounds may be conjugated, either chemically or by genetic engineering, to the antibodies according to the present invention, so as to enhance or provide additional properties to the antibodies, especially properties, which enhance the antibodies' ability to promote alleviation of harmful effects mediated by the interaction between HS and TTR.

In some aspects the present invention relates to a diagnostic method for prediction and/or early diagnostics of TTR amyloidosis. In some embodiments, anti-TTR antibodies may be used to detect the native TTR concentration in the plasma or spinal cord fluid of a patient suspected to suffer or at risk of suffering from TTR amyloidosis such as FAP, familial cardiac amyloidosis and SSA. At present, the cause of TTR amyloidosis is not well understood. Without being limited to any theory, one possible cause of SSA may be increased production of TTR in liver and release into plasma. In some other embodiments, the anti-TTR antibodies, especially the ones binding selectively to the amino acids 24-35 of TTR depicted in SEQ ID NO. 1 or to amino acids 50-61 or 10-24 of SEQ ID NO. 1, or any combinations thereof, may be used to detect the TTR monomer concentration in the plasma or spinal cord fluid of a patient suspected to suffer or at risk of suffering from TTR amyloidosis. Generally, the native TTR tetramer is not considered as pathological. Again without being limited to any theory, the native tetramer is believed to become dissociated, possibly triggered by low pH, resulting in monomers exposing their cryptic sites composed of amino acids 24-35 of SEQ ID NO. 1. These cryptic sites may then interact with HS leading to pathological monomer aggregation. Increased concentration of native TTR tetramers as well as increased concentration of TTR monomers may thus be used for early diagnosis of TTR or predicting an outbreak of TTR.

Concentration of TTR tetramers or monomers in a bodily fluid such as plasma or spinal cord fluid may be determined by conventional methods known in the art. For instance, anti-TTR antibodies, especially those binding to amino acids 24-35 of TTR depicted in SEQ ID NO. 1 or to amino acids 51-60 or 10-24 of SEQ ID NO. 1, or any combinations thereof, may be used for ELISA assays, or other immunological techniques for detection of monomeric TTR. Further, a combination of anti-HS and anti-TTR antibodies may be used to detect a complex of TTR-HS. This may be achieved e.g. by a commercially available Duolink technique by detecting the interaction/complex of HS and TTR preferably in plasma or spinal cord fluid, or in fixed tissues or cells. Other suitable methods are available to a person skilled in the art.

In some embodiments, the anti-TTR antibody may be labeled, either chemically or by genetic engineering, to provide detectable antibodies. Such labeled antibodies may be used in the diagnosis of TTR and are useful tools for imaging purposes.

A further aspect of the present invention relates to a kit for use in the prediction and/or early diagnostics of TTR amyloidosis, comprising one or more reagents for assessing the amount of TTR in a bodily fluid, such as plasma or spinal cord fluid, of a patient suspected to suffer or at risk of suffering from TTR amyloidosis. TTR, the amount or concentration of which is to be assessed, may be either in the form of TTR tetramers or monomers. The reagent to be used in the assessment may be an anti-TTR antibody according to any embodiment described herein, especially a monoclonal antibody binding to amino acids 24-35, amino acids 51-60, amino acids 10-24, or any combinations thereof, of TTR depicted in SEQ ID NO. 1. The kit further comprises one or more reagents for detecting the binding of said antibody to TTR, especially monomeric TTR, if present in the bodily fluid, e.g. by immunological methods, radiation or enzymatic methods, or other methods known in the art. In some embodiments, the kit comprises means for detecting binding of said antibody, if any, by an ELISA assay. In some further embodiments, the kit may comprise an anti-HS antibody for detecting a complex of TTR-HS. Any of the above components may be provided as immobilized on a solid support, such as a microtiter plate.

The following examples are given to further clarify the invention in more detail but are not intended to restrict the scope of the present invention. Further applications and uses are readily apprehended by a person skilled in the art, i.e., clinicians familiar with inflammatory disorders and treatment thereof.

### EXAMPLES

It will be obvious to a person skilled in the art that, as the technology advances, the inventive concept can be implemented in various ways. The invention and its embodiments are not limited to the examples described below but may vary within the scope of the claims.

### Materials and Methods

### Aggregation of TTR

Recombinant wild-type TTR was expressed in bacteria and purified as described previously (Furuya et al. (1991) Biochemistry 30:2415-2421). The purified protein was dissolved in 20 mM phosphate buffer-citric acid (pH 2.7, 3.5, 4.5 or 7.4) to a final concentration of 50 μM for Thioflavin T fluorescence or 500 μM for native-PAGE. Heparin (3H-labeled or unlabeled) and HS were added to the TTR solution to the concentrations as indicated in the respective figure legend. The samples were incubated at 37° C. for the time period as specified.

### TTR Peptides

Synthesized peptides corresponding to different segments of TTR were purchased from commercial resources or synthesized by an expert. The purity and sequence of the peptides were validated by MS/MS analysis.

### Heparin and HS

Heparin has an average molecular mass of 14 kDa as analyzed by gel chromatography on a Superose 12 column (GE Healthcare Biosciences). The HS samples were isolated from porcine tissues and characterized by RPIP-HPLC after enzymatic cleavage with HS/heparin lyases. HS I contained 5% the tri-sulfated disaccharide (IdoA2S-GlcNS6S) species and HS II contained 45% of the species. The 3H-labeled heparin was prepared by N-deacetylation of the heparin followed by re-N-acetylation with N-[3H]acetic anhydride. The heparin oligosaccharides were generated by partial deaminative cleavage of the heparin followed by reduction with NaBH4. The fragmented heparin samples were collected after separation on a Bio-gel P-10 column (Bio-Rad). The amount of saccharides was quantified by carbazole reagent reaction.

### Detection of TTR Aggregates

Thioflavin T (ThT) Fluorescence Assay—

After incubation, 5 μl of the sample was mixed with ThT in water to a final concentration of 20 μM ThT. The mixture was transferred to a flat bottom-black 96-well plate (Grainer) and the fluorescence was measured at excitation- and emission wavelength of 440 and 480 nm, respectively (Gain=55, FARcyte instruments). Samples aggregated at different pHs were adjusted to neutral pH after completed incubation prior to ThT staining. The experiments were run in duplicate or triplicate.

Native-PAGE—

After incubation, 10 μl of the samples was mixed with 2.5 μl of a 5× sample buffer (0.3 M Tris-HCl, pH 6.8, 0.05% bromophenol blue, 50% glycerol) and loaded onto a 10% polyacrylamide gel without SDS. After electrophoresis at 100 V for 2.5 h, the gel was stained with Coomassie blue and scanned. For detection of heparin, the gel was stained with Alcian blue. For quantification of ³H-labeled heparin, the corresponding lanes in the gel were cut into six sections including the stacking gel (see FIG. 4A) that were transferred to a scintillation vial in 3 ml of H₂O. After 2 hr incubation, 2 ml of scintillation cocktail was added and the radioactivity was measured.

### Surface Plasmon Resonance (SPR) Analysis

Interaction of TTR with heparin/HS was evaluated using a Biosensor system (Biacore2000). TTR was dissociated in acidic buffer (pH 2.7) and then diluted into a 50 mM NaAc (pH 5.0) coupling buffer and immobilized onto a CM5 sensor chip to a level of 3000 RU using an amine-coupling kit (GE Healthcare, Uppsala, Sweden). For binding and kinetic analysis, heparin or HS was injected (concentrations indicated in figure legend) over the surface and equilibrated at different pH. The surface was regenerated after each sample with a 30 s wash step of 0.5 M NaCl. All obtained sensorgrams were double-reference subtracted in Biacore evaluation software 2.0, using the reference flow cell and buffer blank sample.

### TTR Aggregation in Cell Culture

Wild-type Chinese hamster ovary (CHO-WT) and HS-deficient CHO-pgsD-677 cells were cultured in F12 medium (Gibco) supplemented with 10% fetal bovine serum (FBS), penicillin/streptomycin (60 and 50 μg/ml, respectively). The cells were seeded at a density of 6000 cells/well in an 8-well LabTec chamber (Nunc) and cultured for 48 h. TTR was dissociated in acidic buffer (pH 2.7) and neutralized with NaOH to neutral pH prior adding to the cells (final conc. 5 μM), which were cultured in starvation media (0.1% FBS). After 48 h, medium was removed and cells were washed extensively in PBS. The cells were thereafter lysed with 1% NaOH. The lysate was centrifuged (10 min at 10000 rpm) and the supernatant was mixed with ThT in water to a final concentration of 20 μM ThT. The fluorescence was measured as described above. Cells cultured without the addition of TTR were used as control to subtract background fluorescence. For immunocytostaining, the cells were fixed in ice cold 95% ethanol and 5% acetic acid and washed with PBS. Cells were permeabilized with 0.4% TritonX-100 and incubated with anti-TTR antibody (1 μg/ml, HPA002550, Atlas Antibodies) followed by incubation with Alexa flour 488 anti-rabbit IgG (8 μg/ml, Molecular probes) for 1 h. The slides were mounted with Vectashield (Vector Labs) containing DAPI counterstain. The TTR immunosignal was captured with Carl Zeiss, AxioCam instrument.

### Detection of TTR Deposition in Cardiac Tissues

Formalin fixed, paraffin embedded sections (15 μm) of cardio-myopathic and control cardiac tissue were deparaffinized in xylene (30 min) and rehydrated at 10 min intervals through ethanol baths (99.9%-70%). For Congo red staining, sections were first incubated in saturated NaCl solution (80% ethanol/0.1% NaOH; 30 min) and then transferred to a filtered 0.4% Congo red (Sigma Aldrich) saturated NaCl solution (30 min). For staining of the sulfated glycosaminoglycans sections were first incubated in 95% ethanol/10% AcOH (1-2 min) and then stained with sulfated Alcian blue (0.45% Alcian Blue 8GX Sigma Aldrich) solution (0.45% Na2SO4/45% ethanol/10% AcOH; 45 min). For immumostaining of TTR and HS, the sections were heated in microwave for antigen retrieval and then permeablized in 0.4% Triton X-100 (15 min). Primary antibody incubations were carried out overnight at 4° C. (anti-TTR: as described above; anti-HS, 1:250 10E4: gift from Dr. Guido David, Center for Human Genetics, University of Leuven, Belgium). For fluorescent detection, sections were incubated with 4 μg/ml of the relevant secondary Alexa fluor 488 or 594 (Molecular probes, USA) antibodies for 30 min at room temperature; nuclei were counterstained with DAPI. For chromogenic detection, the relevant Mach 3 Alkaline Phosphatase Polymer detection kit (Biocare Medical) was developed with Vulcan Fast Red (Biocare Medical). Fluorescent and bright field images were captured with a Nikon DXM1200F instrument (Nikon, Melville, N.Y., USA).

### Analysis of Transgenic Drosophila TTR-Model

The transgenic Drosophila melanogaster strain (Dhet TTR-A) overexpressing a mutant TTR was generated as described (7). TTR-transgenic (w; GMR-Gal4/+; UAS-TTR-A/+) and non-TTR transgenic control flies (w; GMR-Gal4/+; +/+) were used in the study. The TTR expression is under the control of GMR-Gal4 driver, directing TTR expression to the photoreceptor cells during the eye developing stage. The animals were cultivated at room temperature on standard mashed-potato/yeast/agar media. Eggs were reared up with the standard food supplemented with either heparin (Kabivitrum AB, Sweden) or low molecular weight heparin (Klexane®) 10 mg/ml that was mixed with the media. After 9-17 days, the animals were collected for analysis. For ThT-staining, 15 heads of each group were homogenize in 75 μl 0.15 M NaCl. After homogenization, the samples were centrifuged (10 min at 10,000 rpm) and resulting supernatants were mixed with ThT (final concentration of 20 μM ThT). The fluorescence was measured as described above. Non-TTR-transgenic flies were used as control to subtract background fluorescence. For immunostaining, cryostat sections (15 μm) prepared from heads of Drosophila flies (10-14 days of age) were air-dried and fixated in PFA (4%). After washing in PBS, the sections were dehydrated at 10 min intervals through methanol baths (25%-100%). H₂O₂—treatment (2%) was followed by rehydration through methanol baths and the sections were blocked with 5% BSA in PBT (0.2% Triton-X 100). Primary antibodies (anti-TTR and anti-HS/heparin, as described above) were incubated over night in blocking solution. For fluorescent detection, sections were incubated with 2 μg/ml of the relevant secondary Alexa fluor 555 and 633 antibodies for 1 hr in blocking solution. After washing in PBS, the sections were stained with DAPI in mounting media. Images were captured with an inverted confocal microscope (LSM 700, Carl Zeiss), using a PlanApochromat 20×/0.16 or a C-Apochromat 63×/1.2 objective.

### Results

### Co-Deposition of HS and TTR Amyloid in the Cardiomyopathic Heart

Congo red staining of 15 μm thick sections of the heart from a patient diagnosed with cardiomyopathy revealed apple-green birefringence when viewed under polarized light (FIG. 1A), demonstrating amyloid deposition in the specimen. Sulfated Alcian blue staining of the adjacent section revealed a pattern that resembles the Congo red positive morphology (FIG. 1B). Double immunostaining confirmed that TTR deposition is largely co-localized with HS (FIGS. 1C, D and E). In comparison, staining of heart sections from an age-matched control did not produce immunosignals for TTR (FIG. 1F). The codeposition of TTR and HS was prominent in the extracellular space and appeared tightly associated with myocyte membranes, whereas the co-deposition in the vasculature was more variable with some vessel walls lacking HS positive staining (FIGS. 1C and E). No TTR deposition or HS accumulation was detected in the control tissues (FIG. 1F).

### Heparin and HS Promote Fibrillization of WT TTR

Thioflavin T fluorescence analysis revealed that the recombinant human WT TTR expressed in a bacterial system exhibited very low spontaneous fibrillization upon incubation at 37° C. for 48 h, even at low pH (2.7) condition (FIG. 2). However, addition of heparin (an analogue of HS) at this condition prompted TTR aggregation, forming substantial amounts of amyloid-like fibrils (FIG. 3A). Incubation of TTR with heparin-derived oligomers revealed a size dependence effect of heparin, as fragments (12-mer and ti-mer) less than 18-sugar residues had considerably lower effects than longer polymers. Notably, TTR incubated with an 18-mer resulted in more amyloid-like fibrils compared to the TTR incubation with full-length heparin (˜50-mer). HS with higher degrees of sulfation (HS II) enhanced the fibrillization of TTR effectively, albeit to a lesser degree compared to heparin, whereas low sulfated HS (HS I) had a marginal effect on TTR aggregation (FIG. 3B).

Analysis of the TTR incubated with or without heparin by native-PAGE showed that the TTR-heparin aggregate displayed a different migration pattern. In contrast to the continuous smear appearance of the TTR aggregates without heparin, co-incubation with heparin resulted in substantial macromolecular TTR aggregates migrating in the top of the gel, apart from the non-aggregated band observed prior to incubation, representing mono-, di- and tetramers (FIG. 3C). This effect of heparin on TTR aggregation is more pronounced at higher concentrations, suggesting a dose-dependent effect. The TTRheparin incubation generated Congo red positive fibrils as visualized under fluorescence and polarized light microscopy (FIG. 3D), while the TTR incubated without heparin was Congo red negative.

To determine whether heparin was integrated/co-fibrillized into the resulting TTR fibrils or only acted as a catalyst for the fibrillogenesis, TTR was incubated with a mixture of ³H-labeled and unlabeled heparin. The samples of co-incubated heparin-TTR and heparin alone were separated on native-PAGE. Alcian blue staining visualized a heparin migration pattern that was indistinguishable between the two samples (FIG. 6A). Analysis of ³H-labeled heparin by counting the radioactivity in the gel sections (cut as illustrated in FIG. 6A) show that heparin incubated alone was mainly recovered in sections 4, 5 and 6 (FIG. 6B), in accord with the Alcian blue staining. In contrast, heparin co-incubated with TTR was substantially recovered in section 1 (the stacking section), in addition to sections 4, 5 and 6. Notably, Alcian blue failed to stain the heparin in section 1 for the heparin-TTR co-incubated sample, possibly due to a masking effect by the resulting TTR amyloid-like fibrils.

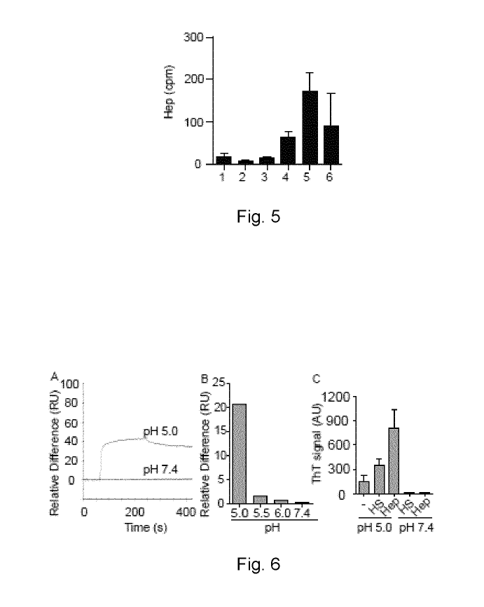

To verify that the heparin recovered in section 1 is not a result of heparin interacting with aggregated TTR, 3H-labeled heparin was incubated with pre-aggregated TTR. Analysis of the product by the same method showed a distribution pattern of ³H-labeled heparin (FIG. 5) that it is almost identical to that of heparin alone (FIG. 4B), indicating that no or very little heparin interacted with aggregated TTR.

### Characterization of TTR-Heparin/HS Interaction

To further investigate the mechanism for the TTR-heparin/HS interaction, we applied surface plasmon resonance (SPR) spectroscopy. Dissociated TTR was immobilized onto a Biacore sensor chip and heparin was allowed to interact with the ligand at neutral (pH 7.4) and mildly acidic (pH 5.0) conditions. Heparin binding to dissociated TTR was observed in mildly acidic pH, but not in the neutral condition, demonstrating a pH-dependency for the interaction (FIG. 6A). The pH-dependent interaction was also observed for HS II (a HS species with higher sulfation degree) that displayed substantial binding at pH 5.0, but significantly reduced binding at pH 5.5 (FIG. 6B). The SPR spectroscopy analysis also showed that sulfation degree of HS/heparin plays a role in this interaction, as affinity of TTR for heparin is 10-fold higher than for HS II, and more than 1000-fold higher than for the low sulfated HS I (FIGS. 7A, B and C). This pH and sulfation degree dependent interaction was further confirmed by measuring fibril formation of TTR incubated with heparin and HS II at pH 5.0 and 7.4 for 7 days (FIG. 6C).

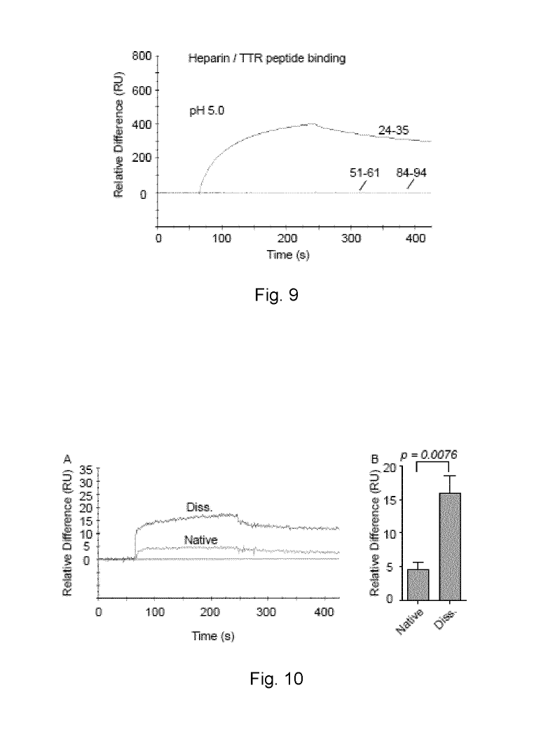

To characterize the TTR-heparin interaction, we incubated TTR and its peptide fragments with ³H-heparin. The binding was analyzed by a nitrocellulose-filter trapping method (18). Among the 16 peptides tested, only the peptide of aa 24-35 (PAINVAVHVFRK) bound to heparin (FIG. 8B). This heparin-binding peptide contains three basic amino acids, including a histidine that likely contributed to the pH dependency seen in the TTR-heparin/HS interaction. This selective binding propy was further verified by SPR analysis (FIG. 9). The 24-35 peptide containing the V30M mutation, which is associated with familial amyloid neuropathy, showed substantially lower binding to heparin compared to the WT peptide (FIG. 8B).

From the binding assay, we noticed that heparin preferentially bound to dissociated TTR (FIG. 8B). For verification, we immobilized the dissociated and native TTR on to Biacore chips and injected HS II over the surface at pH 5.0. The obtained sensorgrams revealed stronger binding of HS II to dissociated TTR (FIG. 10). These results suggest that the heparin/HS binding domain of TTR is cryptic in the natively folded protein.

### HS/Heparin Promotes Fibrillization of TTR in Cell Culture and In Vivo

To investigate whether the effect of HS/heparin in TTR fibrillization is also applicable to cells, the dissociated TTR was incubated with cultures of Chinese hamster ovary WT (CHO-WT) and HS-deficient (CHO-pgsD-677) cells (19) for 48 h. After incubation the cells were washed extensively in PBS to remove free TTR. ThT fluorescence analysis of the cell lysates revealed substantial amounts of amyloid-like fibrils in the CHO-WT cells compared to that in the HS-deficient CHO-pgsD-677 cells (FIG. 11A), demonstrating an effect of HS on TTR fibrillization. Immunocytostaining of the cells with anti-TTR antibody detected trace amounts of positive immunosignals in the HS-deficient cells (CHO-pgsD-677); in comparison, strong positive signals were found in CHO-WT cells (FIG. 11B).

To evaluate the effect of heparin on TTR fibrillization in vivo, we employed a transgenic Drosophila model that expresses an engineered mutant form of human TTR (16, 20). The transgenic flies were reared on standard media supplemented with or without heparin or low molecular weight heparin (Klexane©). ThT fluorescence analysis of the fly head lysate extracted in saline (see Methods) showed that flies fed with heparin supplement generated ThT-positive amyloid-like fibrils, whereas flies given no additive or Klexane© did not generate any fibrils (FIG. 12A). To corroborate the observation of substantial fibril formation in the fly head fed with heparin, the head lysate was analyzed by dot-blot stained with Alcian blue (FIG. 12B). Strong staining in the head lysate from flies fed with heparin was observed, whereas no staining was detected in the corresponding samples from control- or Klexane fed flies. Immunohistostaining of sections prepared from the head of TTR-transgenic flies cultured on heparin-supplemented medium revealed TTR- and heparin co-staining in the retina (FIG. 15D; E, 3D-view of TTR and heparin co-deposited material). Neither sections prepared from transgenic flies reared on regular medium (FIG. 15F), nor sections prepared from nontransgenic flies reared on heparin-supplemented medium (FIG. 15G), showed co-staining for TTR and heparin in the retina.

1. A method of diagnosing TTR amyloidosis, comprising the steps of:
(a) providing a sample of a bodily fluid from a human subject,
(b) determining the concentration of TTR in said bodily fluid,
(c) determining said human subject as suffering from or at high risk of developing TTR amyloidosis, if the TTR concentration determined in step (b) is higher than in a control sample.

2. The method according to claim 1, wherein said bodily fluid is selected from a group consisting of plasma and spinal cord fluid.

3. The method according to claim 1 or 2, wherein said TTR in the bodily fluid is in the form of monomers.

4. The method according to claim 1 or 2, wherein said TTR is in the form of a tetramer.

5. The method according to claim, comprising a further step between steps a) and b), wherein said sample is exposed to low pH, such as pH 2.7, in order to dissociate the TTR tetramer into monomers.

6. The method according to any one of claims 3 to 5, wherein the concentration of TTR is determined by using an anti-TTR antibody.

7. The method according to claim 6, wherein said antibody binds selectively to amino acids 24-35, 50-61, or 10-24 of TTR depicted in SEQ ID NO. 1, or to any combinations thereof.

8. The method according to any one of claims 1 to 7, wherein said TTR amyloidosis is selected from a group consisting of familial amyloid polyneuropathy, familial cardiac amyloidosis, and sporadic senile systemic amyloidosis.

9. A kit for use in the method according to any one of claims 1 to 8, comprising
a) an anti-TTR antibody, and
b) one or more reagents for detecting the expression level of TTR in a sample of a bodily fluid.

10. The kit according to claim 9, further comprising an anti-HS antibody.

11. The kit according to claim 9 or 10, wherein said one or more reagents are selected from the group consisting of secondary antibodies; and colorimetric, chemiluminescent or chemifluorescent substrates for alkaline phosphatase (AP), horseradish peroxidase (HRP) or other enzyme reporter systems.

12. A method of preventing, alleviating and/or treating transthyretin (TTR) amyloidosis in a patient in need thereof by administering an efficient amount of an agent capable of interfering with the interaction of TTR and heparan sulfate (HS).

13. The method according to claim 12, wherein said agent is selected from a group consisting of heparan sulfate oligosaccharides, heparan sulfate polysaccharides, non-anticoagulant heparin oligosaccharides, non-anti-coagulant heparin polysaccharides and mimetics thereof.

14. The method according to claim 12, wherein said agent is a TTR peptide comprising an amino acid sequence depicted in SEQ ID NO. 1 or a fragment thereof.

15. The method according to claim 12, wherein said agent is an anti-TTR antibody.

16. The method according to any of claims 12 to 15, wherein said TTR amyloidosis is selected from a group consisting of familial amyloid polyneuropathy, familial cardiac amyloidosis, and sporadic senile systemic amyloidosis.

17. An agent capable of interfering with the interaction between HS and TTR for use in the prevention, alleviation and/or treatment of TTR amyloidosis.

18. The agent according to claim 17, wherein said agent is selected from a group consisting of heparan sulfate oligosaccharides, heparan sulfate polysaccharides, non-anticoagulant heparin oligosaccharides, non-anti-coagulant heparin polysaccharides and mimetics thereof.

19. The agent according to claim 17, wherein said agent is a TTR peptide comprising an amino acid sequence depicted in SEQ ID NO. 1 or a fragment thereof.

20. The agent according to claim 17, wherein said agent is an anti-TTR antibody.

21. The agent according to any of claims 17 to 20, wherein said TTR amyloidosis is selected from a group consisting of familial amyloid polyneuropathy, familial cardiac amyloidosis, and sporadic senile systemic amyloidosis.

22. A pharmaceutical composition comprising an agent capable of interfering with the interaction between HS and TTR, and a pharmaceutically acceptable carrier.
